(19) United States
(12) Patent Application Publication
(10) Pub. No.: US 2024/0277223 A1
(43) Pub. Date: Aug. 22, 2024

(54) Testing System

(71) Applicant: University of Strathclyde, Glasgow (GB)

(72) Inventors: Mario E. Giardini, Glasgow (GB); Guillermo Vivas-Mateos, Glasgow (GB)

(21) Appl. No.: 18/292,124

(22) PCT Filed: Jul. 25, 2022
(86) PCT No.: PCT/GB2022/051948

§ 371 Date: Jan. 25, 2024

(30) Foreign Application Priority Data
Jul. 26, 2021 (GB) 2110698.4

(51) Int. Cl. A61B 3/06; A61B 3/00

(52) U.S. Cl. CPC A61B 3/066; A61B 3/005; A61B 3/0058

(57) ABSTRACT

A computer implemented testing system for testing perception of a user, the testing system being configured to control at least one visual display system to: display one or more items against a background, wherein at least one display property of the one or more items differs from that of the background and at least one other display property of the one or more items is the same as that of the background; and vary the at least one display property of the one or more items; or display different items of the one or more items wherein the at least one display property differs between different items. A point at which the user ceases to be able to distinguish the one or more items from the background can be identified and the at least one display property of the at least one item being displayed at that point can be determined and used to provide an indication of the user's ability to perceive or sensitivity to the at least one display property. Also described are a corresponding method and computer program.

### FIELD

The present disclosure relates to a testing system and associated method, particularly a vision testing system for testing for properties of a user's vision.

### BACKGROUND

Red desaturation perception (or ‘red saturation perception’) is the capacity to distinguish red from grey. This can be diminished in people suffering from problems of the optic nerve. Examples of such problems include optic neuritis (a disease related to multiple sclerosis), inflammation, raised intracranial pressure, inherited neuropathies, ischaemic events, compression, tumour infiltration, amongst others. As such, a reliable measure of a user's red saturation can be used to identify, quantify, and perhaps provide early warning of, any such problems of the optic nerve.

However, current techniques for testing red desaturation perception are qualitative and/or subjective. They do not quantify a degree of saturation perception, or they do so in a subjective way. This leads to significant drawbacks. For example, the present prior art methods do not allow quantitative criteria for evaluation and don't provide for acceptable monitoring of progression of saturation perception over time. The prior techniques do not quantify differences between eyes, differences between patients/test subjects, or differences between test operators.

The current accepted standard test for red desaturation perception testing relies on subjective evaluation of a red object. In the current conventional test, a physician asks the patient to observe a red object (for example, a Coca Cola bottle cap) and to indicate if one eye sees the object more dull (i.e. more grey, more washed out, less intense red, etc.) than the other. However, this test is clearly very qualitative and subjective,

Another test is the Cullen chart. The Cullen chart provides a red disc shape in the centre of the cart, with a plurality of identical red disc shapes around the periphery of the card. The patient is then asked to fix their gaze on the central spot and indicate the number of discs they can see. However, this test is also not quantitative.

Another test uses a red desaturation confrontation disc, which can come as either a physical apparatus or in a digital format. The patient is asked to match the saturation of red targets on a chart or on a screen until they see them with the same saturation. This test is also highly subjective.

It would be beneficial to have a test that is simple, yet capable of providing quantitative analysis without being subjective. It would also be beneficial to have a test that is capable of automation, e.g. implemented by a computer based system.

### Statements

Various aspects of the present invention are defined in the independent claims. Some preferred features are defined in the dependent claims.

According to a first aspect of the present disclosure is a computer implemented testing system configured to control at least one visual display system to display one or more items against a background, wherein at least one display property of the one or more items differs from that of the background and at least one other display property of the one or more items is the same as that of the background. The testing system may be configured to vary the at least one display property of the one or more items or display different items or groups of items of the one or more items wherein the at least one display property differs between different items or groups of items. The testing system may be configured to maintain the at least one other display property of the one or more items to be the same as that of the background, e.g. throughout the test. The testing system may comprise and/or be configured to communicate with the at least one visual display unit.

The display properties (i.e. the at least one display property and/or the at least one other display property) may comprise one or more of: luminance, hue and/or saturation.

The at least one display property may comprise saturation. That is, the saturation of the at least one item may differ from the saturation of the background. The testing system may be configured to vary the saturation of the at least one item. The testing system may be configured to display a plurality of the items wherein the saturation differs between different items of the plurality of items.

The at least one other display property may comprise one or both of luminance and/or hue. The testing system may be configured to maintain or display each of the at least one items with the same luminance and/or hue as the background.

The background may be of a different colour to the one or more items. The items may be red in colour. The background may be grey in colour. In examples, the testing system may be configured such that the only display property that is varied or differs is saturation, e.g. the saturation of the one or more items, which may be red, and wherein the background may be grey.

The at least one item may comprise a plurality of the items, which may be displayed successively or together. The testing system may be configured to display groups of the items. The testing system may be configured to sequentially display items from the plurality of items. The at least one of the plurality of display properties of at least one or all of the items may be different to that of another or the next of the sequentially displayed items. The testing system may be configured to successively reduce the at least one display property e.g. saturation, of successively displayed items. The testing system may be configured to successively increase the at least one display property e.g. saturation, of successively displayed items. The testing system may be configured to switch or alternate between increasing and decreasing the at least one display property, e.g. saturation, of successively displayed items or to switch or alternate between increasing and decreasing the at least one display property, e.g. saturation, of the one or more items with time. Switching or alternating between increasing and decreasing the at least one display property may allow the testing system to hone in on the value of the at least one display property at which the user can distinguish or no longer distinguish between the item and background, which may allow this to be determined with greater accuracy. The testing system may be configured to successively vary the at least one property e.g. saturation, of successively displayed items so as to be closer to or further from the corresponding display property or properties of the background that the last displayed item. The testing system may be configured to successively vary the at least one property e.g. saturation, of items or successively displayed items to fade into the background or appear out of the background or to switch or alternate between fading into the background and appearing out of the background. The at least one other display property of at least one or all of the successively displayed items of the plurality of items may be the same.

The testing system may be configured to vary the at least one property of the at least one item whilst maintaining the at least one other of the plurality of display properties constant. The testing system may be configured to reduce the at least one property (e.g. the saturation) of the at least one item of the one or more items with time whilst maintaining the at least one other of the plurality of display properties (e.g. the luminance and/or the hue) constant. The testing system may be configured to vary the at least one property (e.g. the saturation) of the at least one item with time so as to be closer to the corresponding display property or properties of the background whilst maintaining the at least one other of the plurality of display properties (e.g. the luminance and/or the hue) constant.

The testing system may be configured to provide the successively displayed items or vary the one or more display properties at least until the user is unable to distinguish the displayed item or items from the background. The testing system may be configured to determine a value for the users' ability to perceive the at least one display property comprising or based on the determined value of the at least one display property with which the at least one item is being displayed when the user is unable to distinguish the at least one item displayed from the background.

The testing system may comprise or be configured to communicate with a user input device. The user input device may be configured to receive user input responsive to the displayed items. The testing system may be configured to determine from the user input the at least one display property, e.g. saturation, of an item of the at least one items or a difference in the at least one display property, e.g. saturation, of the item and the corresponding property of the background that the user can perceive or cannot perceive.

The user input device, may comprise an active user input device, which may, for example, be configured to receive active, deliberate, cognizant or voluntary actions of the user to operate the user input device responsive to the displayed item or items. Examples of active user input devices may include a button, a keyboard, a touch screen, a joystick, a trackball, a mouse or other suitable user input device that is actively operated by the user in response to the displayed item or items. Alternatively or additionally, the user input device may comprise a passive user input device, which may be configured to receive or determine passive, involuntary, non-cognizant or non-deliberate actions of the user in response to the displayed item or items. Examples of passive user input devices include a head or eye motion tracker which may optionally be integrated into a headset, one or more accelerometers or 3DOF accelerometers, one or more gyroscopes, one or more magnetometers, one or more video cameras for capturing still and/or video images of the user, and/or the like.

The testing system may comprise or be configured to communicate with at least one user input device. The user input device may be being configured to receive user input indicative of the user's perception of the displayed items. The testing system may be configured to identify when the user input indicative of the user's perception of the displayed items differs from, or starts to match or become correlated with, the item or items being displayed. That is, in examples where the at least one property becomes more different to that of the background over time (i.e. the at least one item appears out of the background) then the testing system may be configured to identify when the user input indicative of the user's perception of the displayed items starts to match or become the correlated with the item or items being displayed. In examples where the at least one property becomes closer to that of the background over time (i.e. the at least one item fades into the background) then the testing system may be configured to identify when the user input indicative of the user's perception of the displayed items differs from the item or items being displayed. The testing system may be configured to determine the value of the at least one display property with which the at least one item is being displayed when it is identified that the user input indicative of the user's perception of the displayed items differs from, or starts to match or become correlated with, the items being displayed. The testing system may be configured to provide a value for the users' ability to perceive the at least one display property, the value comprising or being based on the determined value of the at least one display property with which the at least one item is being displayed when it is identified that the user input indicative of the user's perception of the displayed items differs from, or starts to match or become correlated with, the items being displayed. The value for the users' ability to perceive the at least one display property may be or comprise a user perception limit of the at least one display property, e.g. of saturation. One or more or each of: the determination of when the movement of the user's eye and/or head ceases or begins to be correlated with the position and/or movement of the movement of the at least one item; the determination of the at least one display property with which the at least one item is being displayed when the position and/or movement of the user's eye and/or head ceases or begins to be correlated with the position and/or movement of the movement of the at least one item; and/or the determination and/or provision of the value for the users' ability to perceive the at least one display property may be performed offline and/or not during the test and/or by a remote computer that is remote from the user input device that is optionally connected to the user input device via a wide area network

The at least one item may comprise letters, numbers or other characters or optotypes. Different items of the plurality of items may be or comprise different letters, numbers or other characters or optotypes. The user input device may be configured to receive user input of the letter, number other character or optotype perceived by the user. The testing system may be configured to determine errors in, or differences between, the input the letter, number other character or optotype and the displayed letter, number other character or optotype perceived by the user. The testing system may be configured to determine that the user input indicative of the user's perception of the displayed items differs from the items being displayed when the user makes an error or a threshold number of errors in inputting the letter, number other character or optotype of the displayed item or items.

The items may comprise shapes, e.g. dots or circles. The items may be of differing shapes. The user input device may be configured to receive user input indicative of the user's perception of the shapes being displayed, e.g. indicative of a number of shapes, or of the types of shapes, or of the size of shapes, or of the location of the shapes, or of the presence of movement of the shapes or of the speed of movement of the shapes, or of changes in the shapes, a change in size of the shapes, and/or the like. The testing system may be configured to determine that the user input indicative of the user's perception of the displayed items differs from the items being displayed when the user makes an error or threshold number of errors in inputting or providing an indication responsive to the number, size, speed, location, change, movement and/or shape of the displayed shape or shapes.

The testing system may be configured to move at least one item on the display system. The user input device may be configured to receive user input, e.g. via a touch screen, mouse, pointer, trackpad, light pen or other user input device. The user input may be indicative of where the user believes the at least one item to be (i.e. the perceived location of the at least one item) and/or what the user believes the motion of the at least one item is. The testing system may be configured to determine that the user input indicative of the user's perception of the displayed items differs from the items being displayed when the user input becomes uncorrelated with the position of the at least one item, e.g. at least for a threshold period of time.

The user input device may comprise a tracking system, which may comprise an eye and/or head movement tracker for tracking movement of an eye and/or head of the user. The tracking system may be configured to determine when the movement of the user's eye and/or head ceases or begins to be correlated with the movement of the position and/or movement of the at least one item. The testing system may be configured to determine that the user input indicative of the user's perception of the displayed items differs from the items being displayed when the user's eye and/or head movement becomes uncorrelated with the position and/or movement of the at least one item, e.g. at least for a threshold period of time. The testing system may be configured to determine that the user input indicative of the user's perception of the displayed items matches the items being displayed when the user's eye and/or head movement becomes correlated with the position and/or movement of the at least one item, e.g. at least for a threshold period of time.

The analysis of when the user input collected by the user input device may be in real time, on-line, on the fly and/or during the performance of the test by the testing system. The analysis of when the user input collected by the user input device may be retrospective, off-line, and/or after the performance of the test by the testing system.

The visual display system may comprise a headset display. The visual display system may comprise a monitor or visual display unit. The visual display may comprise a touch screen. The visual display may include a projector. The visual display may include at least one of: a holographic display, an autostereoscopic display, or another type of three-dimensional display. At least one of the user input devices, such as one or more of: the motion tracker, the accelerometer, the gyroscope, and/or the magnetometer may be comprised in the headset display.

The testing system may be configured to identify a condition of the user based on the value for the users' ability to perceive the at least one display property, e.g. if the value for the users' ability to perceive the at least one display property is above or below a threshold or is within a range. The condition may be a condition of the eye or optic nerve, such as optic neuritis, a disease related to multiple sclerosis, inflammation, raised intracranial pressure, inherited neuropathies, ischaemic events, compression, tumour infiltration, of a part of the brain that deals with vision or visual stimuli, and/or the like.

The testing system may comprise or be configured to implement a colour model defining at least one or each of the display properties of the item. The colour model may define the display properties based on red, green and blue values for the colour. The colour model may define at least one or each of the display properties of the item based on a baseline grey level or a grey level of the background modified by altering at least one other of the display properties. The colour model may define the saturation of the item based on a baseline grey level modified by altering one or two of: the red, green, and blue values for the colour.

The testing system may be implemented using a processing device, which may be a mobile or network enabled device. The device may be or comprise or be comprised in a mobile phone, smartphone, PDA, tablet computer, laptop computer, and/or the like. The controller or processing system may be implemented by a suitable field programmable gate array (FPGA) or complex logic programmable device (CPLD) or application-specific integrated circuit (ASIC). The controller or processing system may be implemented by a suitable program or application (app) running on the device. The device may comprise at least one processor, such as a central processing unit (CPU), maths co-processor (MCP), graphics processing unit (GPU), tensor processing unit (TPU) and/or the like. The at least one processor may be a single core or multicore processor. The device may comprise memory and/or other data storage, which may be implemented on DRAM (dynamic random access memory), SSD (solid state drive), HDD (hard disk drive) or other suitable magnetic, optical and/or electronic memory device. The at least one processor and/or the memory and/or data storage may be arranged locally, e.g. provided in a single device or in multiple devices in in communication at a single location or may be distributed over several local and/or remote devices. The device may comprise a communications module, e.g. a wireless and/or wired communications module. The communications module may be configured to communicate over a cellular communications network, Wi-Fi, Bluetooth, ZigBee, near field communications (NFC), IR, satellite communications, other internet enabling networks and/or the like. The communications module may be configured to communicate via Ethernet or other wired network or connections, via a telecommunications network such as a POTS, PSTN, DSL, ADSL, optical carrier line, and/or ISDN link or network and/or the like, via the cloud and/or via the internet, or other suitable data carrying network. The communications module may be configured to communicate via optical communications such as optical wireless communications (OWC), optical free space communications or Li-Fi or via optical fibres and/or the like. The device and/or the controller or the at least one processor or processing unit may be configured to communicate with the remote server or data store via the communications module. The controller or processing unit may comprise or be implemented using the at least one processor, the memory and/or other data storage and/or the communications module of the device.

According to a second aspect of the present disclosure is a method of operating a testing system, such as the testing system of the first aspect. The method comprises operating the testing system to:
display one or more items against a background, wherein at least one display property of the one or more items differs from that of the background and at least one other display property of the one or more items is the same as that of the background; and
vary the at least one display property of the one or more items; or display different items or groups of items of the one or more items wherein the at least one display property differs between different items or groups of items.
   According to a third aspect of the present disclosure is a computer program product configured such that, when executed by a processing system of a testing system, such as a testing system of the first aspect, causes the testing system to:
display one or more items against a background, wherein at least one display property of the one or more items differs from that of the background and at least one other display property of the one or more items is the same as that of the background; and
vary the at least one display property of the one or more items; or display different items or groups of items of the one or more items wherein the at least one display property differs between different items or groups of items.

The computer program product may be embodied on a tangible, non-transient carrier medium, such as but not limited to a memory card, an optical disk or other optical storage, a magnetic disk or other magnetic storage, quantum memory, a memory such as RAM, ROM, an solid state device (SSD) memory, and/or the like.

The individual features and/or combinations of features defined above in accordance with any aspect of the present invention or below in relation to any specific embodiment of the invention may be utilised, either separately and individually, alone or in combination with any other defined feature, in any other aspect or embodiment of the invention.

Furthermore, the present invention is intended to cover apparatus configured to perform any feature described herein in relation to a method and/or a method of using or producing, using or manufacturing any apparatus feature described herein.

### BRIEF DESCRIPTION OF THE DRAWINGS

These and other aspects of the present invention will now be described, by way of example only, with reference to the accompanying drawings, in which:

FIG. 1 is a schematic of a testing system;

FIG. 2 is a schematic of an alternative testing system;

FIG. 3 is a view of a display of a testing system;

FIG. 4 is a view of an alternative display of a testing system;

FIG. 5 is a view of another display of a testing system;

FIG. 6 is a view of a further display of a testing system; and

FIG. 7 is a flowchart of a method of operation of a testing system.

### DETAILED DESCRIPTION OF DRAWINGS

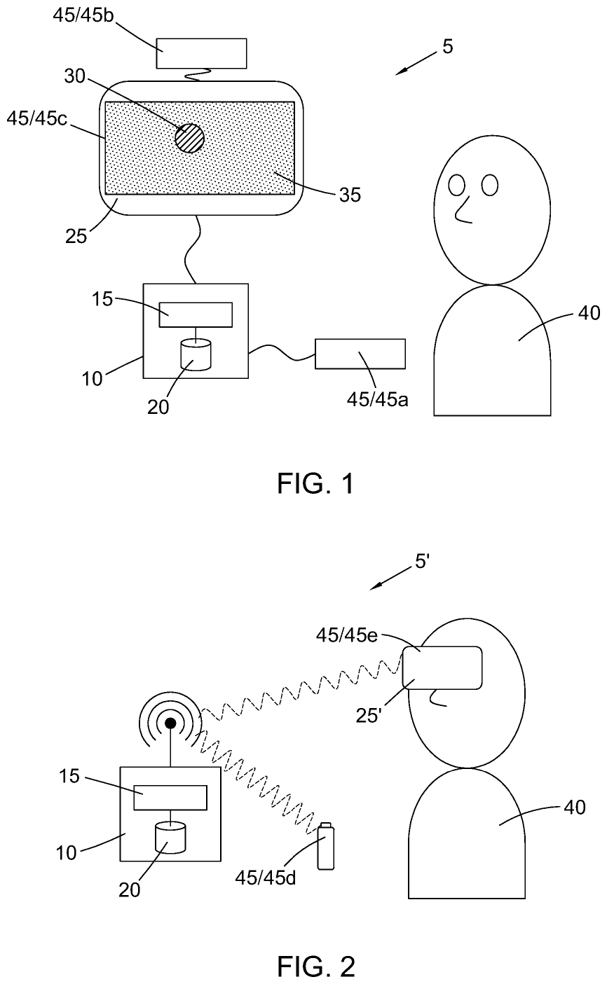

FIG. 1 shows a system 5 for testing user's perception, such as a user's perception of colour. In an example, the system can be used for testing red desaturation perception, but the same principles can be applied to test for other aspects of a user's perception. Beneficially, with at least some examples described herein, it may be possible to avoid or reduce subjectivity in the tests, provide a quantitative assessment of a users' perception and overcome problems in using computer display systems in such tests.

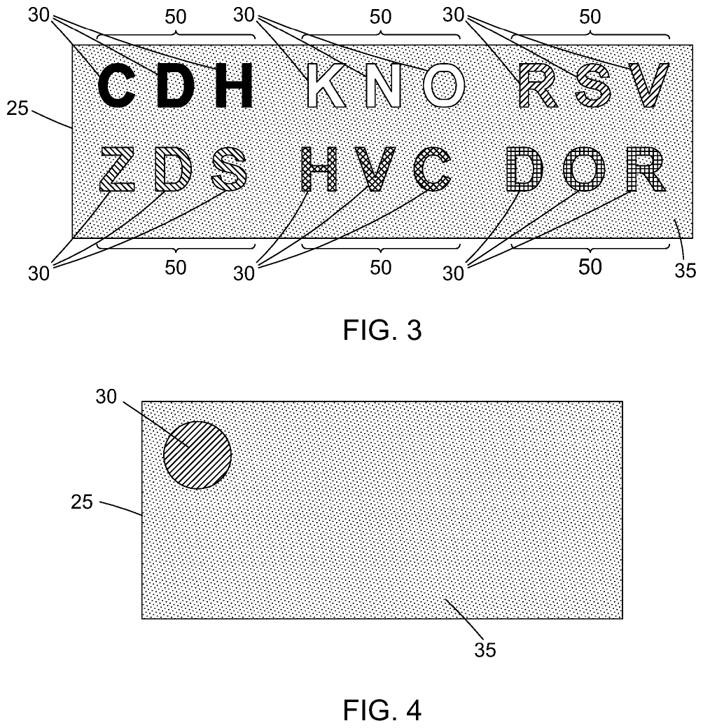

The system 5 of FIG. 1 comprises a processing system 10 having at least one processor 15 and data storage 20, such as ROM, RAM, a magnetic storage device, an optical storage device or a solid state storage device. The system 5 comprises a visual display unit 25, such as a monitor, touch screen or the like, that is connected or in communication with the processing device 10. The visual display unit 25 is operable responsive to the processing system 10 to display content stored in the data storage 20 and/or generated by the at least one processor 15. In particular, the processing system 10 is configured to control the visual display unit 25 to display one or more items 30 (see FIGS. 3 to 6) over a background 35 (also in FIGS. 3 to 6) with one or more display properties such as hue, saturation and/or luminance of the items and the background. In particular, the values of the one or more display properties (e.g. hue, saturation and/or luminance) of the one or more items output from the visual display unit 25 relative to the corresponding values of the one of more display properties of the background are controlled by the processing system 10 in order to perform the perception testing of a user 40 viewing the visual display unit 25.

Beneficially, the system 5 further comprises at least one user input device 45 for receiving input from the user 40 in response to the display of the items 30 on the visual display unit 25. In FIG. 1, the user input device 45 is separate from the processing system 10 and the visual display unit 20, e.g. it may be a keyboard, a button press system having one or more buttons, a trackpad, an eye motion monitor for monitoring or tracking motion of the user's 40 eyes, or the like. In the example of FIG. 1 the at least one user input device 45 comprises a keyboard 45a, a user eye monitor 45b and a touchscreen 45c, but the user input devices are not limited to these and alternative or additional user input devices could be used. The eye monitor 45b could be camera based with associated object recognition, for example. However, in other examples, the user input device 45 may be integrated into one of the processing system 10 or visual display unit 25, e.g. as a touchscreen or the like. The provision of the at least one user input device allows for a fully automated testing system 5 and may provide greater accuracy in quantifying the user's degree of perception of the one or more display properties.

In the example of FIG. 1, the visual display unit 25 is a computer monitor. However, the visual display unit may take other forms.

An alternative system 5′ for testing user's perception is shown in FIG. 2. In the system 5′ of FIG. 2, the visual display unit 25′ is advantageously a headset to be worn by the user 40, such as but not limited to a headset of the type commonly deployed as a “virtual reality” or 3D headset in which the headset is worn over the user's eyes and a separate display or display area is provided to each eye or the user 40. This arrangement allows for better control of ambient light and individual testing of each eye.

Furthermore, in the example of FIG. 1, each of the visual display unit 25 and the user input devices 45a, 45b, 45c communicate with the processing unit via wired connections. However, other communications options could be used and FIG. 2 shows communication between the processing system 10 and the visual display unit 25′ (the headset) and the user input devices 45d, 45e via wireless communication (e.g. via Bluetooth, Wi-Fi, or other suitable wireless communication protocol). However, arrangements described herein, including those of FIGS. 1 and 2, could optionally use wired or wireless communications or a combination of both, depending on the application and other considerations.

The user input devices 45 in the example of FIG. 2 include a user operated button push 45d and at least one sensor 45e integrated into the headset 25′ to monitor movement of the user's 40 eyes and/or of the user's 40 head. The monitoring of the movement of the user's eyes may be performed by using an eye tracker. The monitoring of the movement of the user's head may be performed by using a sensor capable of monitoring head movement, e.g. at least one or more or each from: an accelerometer, a gyroscope, a magnetometer, and/or the like. These sensors can optionally be integrated inside the headset. Alternatively or additionally, the monitoring of the movement of the user's eyes and/or head can be performed, e.g. using computer vision through a digital camera (e.g. a webcam). This digital camera may be integrated into other elements of the system (e.g. may be integrated into a computer monitor, or a phone, or a tablet computer), or can be provided as a separate or stand-alone device.

Although examples of suitable systems 5, 5′ are shown in FIGS. 1 and 2, the present disclosure is not limited to these and other systems comprising at least some form of processing ability, capability for providing some form of visual display responsive thereto, and optionally some form of user input device may potentially be operated using the concepts described in the present disclosure.

Beneficially, the processing system 10 is configured to control the visual display unit 25, 25′ to display the one or more items 30 over the background 35. Both the one or more items 30 and the background are displayed on the visual display unit 25, 25′ such that a plurality of their display properties such as hue, luminance and/or saturation are controlled. Specifically, one or more of the display properties of the one or more items 30 differ to those of the background 35 during the test. For example, the one or more items 30 can be displayed with a different saturation to the background 35 during the test. In addition, the one or more of the display properties of the one or more items 30 can be varied during the test or different items 30 with different saturations, or different luminances, or different hues, can be presented using the visual display unit 25, 25′ during the test.

In addition, one or more other of the display properties of the one or more items 30 can be the same as those for the background 35 during the test. For example, the luminance and optionally the hue at which both the one or more items 30 and the background 35 are displayed using the visual display unit 25, 25′ can be controlled to be the same throughout the test.

In this way, during the test, the user 45 observes the visual display unit 25, 25′. The one or more items 30 are displayed with saturations that are different to the saturation of the background 35. This may involve different items 30 or groups 50 of items being displayed (at the same time and/or successively), with each different item 30 or group 50 of items 30 having a different saturation. Alternatively or additionally, the one or more items 30 may be persistently displayed and the saturation of the one or more items 30 is varied. The user then provides feedback or input based on their perception of the one or more items 30 against the background 35 using the user input device(s) 45 or otherwise.

The example of FIG. 1 may be better for children or other users for whom wearing a headset would be unwelcome. The example of FIG. 2 allows better control over the lighting and environment. Furthermore, the use of the headset in which a different display or part of a display can be provided to different eyes, the individual response with each eye or the difference in response between eyes can be determined more easily, which may be beneficial.

FIG. 3 shows a specific example of a display shown to the user 40, e.g. on the visual display unit 25, 25′ of FIG. 1 or 2. In this example, a plurality of the items 30 are displayed simultaneously, wherein the items 30 are optotypes such as letters or numbers. The items 30 are displayed in a set colour, in this case red, against the background 35 which is in a different set colour, in this case grey. In this example, groups 50 of one or more items 30 (in this case groups 50 of three items 30) are displayed, wherein each item 30 in any given group 50 has the same display properties, i.e. the same hue, saturation and luminance, but represents a different optotype. In examples, such as that of FIG. 1, where the visual display unit 25 is a monitor or similar, the lighting in the room can be controlled to be consistent. In embodiments where the visual display unit 25′ is a headset, such as that of FIG. 2, it is easier to control (or exclude) ambient lighting such that less or no ambient lighting control is required.

The items 30 are all displayed so that they have the same hue as each other, in this case corresponding to pure red. The items 30 are also all displayed so that they have the same luminance as the background 35 throughout the test. However, the items 30 in any given group 50 all have a different saturation to the items in at least one or each of the other group 50 and also to the background 35. The saturation of the background 35 stays constant. Specifically, the saturation of each successive group of items 30 gets closer to the saturation of the background 35, i.e. for each successive group 50 of items 30, the difference between the saturation of the items 30 in that group 50 and the background 35 is less than that of the items 30 in the preceding group 50.

To perform the test, the user 40 reads the items (i.e. the optotypes) either with one or the other eye closed (to distinguish between eyes if a monitor is being used, as in FIG. 1) or with both eyes open (if eye distinction is not important or if the embodiment of FIG. 2 is being used that can provide different displays or selective display to individual eyes via the headset 25′). The user can then identify the displayed optotype associated with each item 30 using the user input device 45, e.g. via the keyboard 45a, or by other means, e.g. orally, in writing, etc. When the user 40 makes an error in identifying an item 30 (i.e. optotype), or when a threshold number of errors in identifying the item has been made, then it is determined that the saturation level associated with that item 30 cannot be perceived. The determined saturation level that the user cannot perceive is provided as, or used to determine, a measure of the user's 40 perception of saturation of that colour (e.g. of red in this case). In some examples, the test can be repeated and an average of the determined saturation levels is determined and used to determine the measure of the user's 40 perception of saturation of that colour. In some examples, the determined level that the user cannot perceive can be used to inform further steps in the test. For example, once established that the user cannot perceive a certain saturation level, the test may proceed by showing the user again a higher saturation level, to refine and/or confirm at which saturation level the perception actually stops.

Variations of the example shown in FIG. 3 are possible. For example, more or fewer items 30 can be included in each group 50. The groups 50 of items 30 may be static, or the groups 50 of items 30 may refresh or move, e.g. by scrolling, changing page, etc. Furthermore, although in the example of FIG. 3 the hue and luminance of the optotypes and background are constant and the saturation changes, any two of the luminance, hue and saturation of the items 30 may be kept constant and the other of the luminance, hue or saturation of the items 30 may be varied, which can be used to test for different conditions. Furthermore, although the items 30 are beneficially presented as red, and the background 35 as grey (which can be particularly beneficial in identifying certain conditions), other colours or colour combinations could be used.

Furthermore, properties such as the number of lines, the optotype size, the spacing, displayed on the monitor, and the like can be defined according to the task and the testing system 5 may be configured to monitor size, along with any other factor influencing readability (such as room lighting, test subject visual acuity, etc.). Similarly, while the example shown in FIG. 3 uses letter optotypes, other optotypes can be used (e.g. Landoldt C, tumbling E, picture-based optotypes), for example to cater for illiterate subjects, or children, or people with cognitive disabilities. Crowding bars (non-optotype symbols) may be present, as they affect character readability.

In other examples, instead of the items 30 comprising optotypes, the items 30 can comprise geometric shapes, such as circles. In the example of FIG. 4, the items 30 are in the form of shapes (a circle is shown). In this example, the items 30 have the same luminance (and optionally the same hue) as the background 35, differing only by saturation. Again, the items 30 are beneficially red and the background can optionally be grey. The items 30 are selected and/or displayed in such a way that the user 40 can provide or input a value that indicates whether they have perceived any, or some, or all of the items 30.

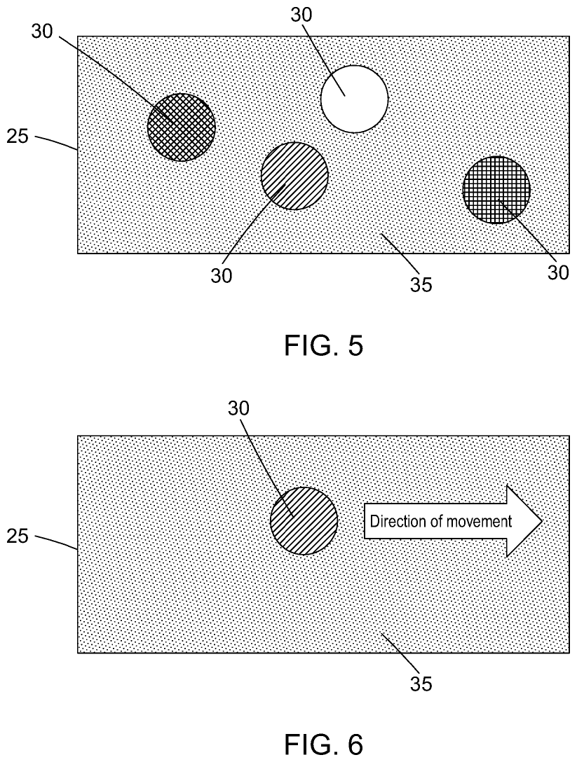

For example, different numbers of items having a particular saturation can be displayed and the user 40 has then to provide the number of items 30 (e.g. using the user input device 45, orally, by writing or otherwise). An example of this is shown in FIG. 5. In this example, a plurality of items 30 in the form of shapes (red circles in the example shown) are displayed on the visual display unit 25, 25′, wherein each of the items 30 has the same luminance (and optionally hue) as the background 35 (the background 35 optionally being grey in the shown example) but a different saturation. Furthermore, at least one or each of the items 30 displayed has a different saturation to at least one or each other of the items 30 displayed. A user can be asked to indicate a number of items 30 that they can perceive. If the number of items perceived by the user is different to the number of items displayed, then the number of items 30 provided by the user 40 can be used to work out a range for the user's perception of the saturation based on the lowest and highest saturations of the items 30 that are perceived or not perceived by the user. Optionally, the system 5, 5′ can be configured to provide a further plurality of items 30 having an assortment of different saturations in the range identified in the previous step in order to refine the range for the user's perception of the saturation. It will be appreciated that further refining steps may be provided to further refine the range of the user's perception of the saturation, e.g. until the range is less than a threshold amount. In another example, one or more of the items 30 are provided as different shapes (e.g. circle, star, square, triangle, etc.) and the user 40 has then to provide the correct shape of the item(s) 30 (e.g. using the user input device 45, orally, by writing or otherwise). In another example, the items 30 having different saturations are shown at different or random time intervals, and the user 40 indicates or inputs when they perceive the each respective item 30 (e.g. using the user input device 45, orally, by writing or otherwise). In a further example, the items 30 having different saturations are shown at different or random positions, and the user 40 indicates or inputs where on the visual display unit 25 they perceive the each respective item 30 (e.g. using the user input device 45, orally, by writing or otherwise, but particularly suited to the user input device 45, 45c being a touchscreen 45c, a mouse, a trackpad or the like).

Again, when the user 40 makes an error in identifying an item 30 over the background 35, or when a threshold number of errors in identifying the item 30 over the background 35 has been made, then it is determined that the saturation level associated with the displayed item 30 that lead to the error or threshold number of errors cannot be perceived. The determined saturation level that the user cannot perceive is provided as a measure of the user's 40 ability to perceive saturation of that colour. In some examples, the test can be repeated and an average of the determined saturation levels is determined as a measure of the user's 40 perception of saturation of that colour.

FIG. 6 illustrates an example in which one or more items 30 are provided so as to move around on the visual display unit 25, 25′. In the example shown, one item 30 is displayed but more items 30 could be displayed in alternative examples. As in the other examples, the luminance of the item 30 is constantly kept the same as the luminance of the background 35. The hues of the item 30 and background 35 are also kept constant, and can be the same or different to each other. In the example shown, the item 30 is kept at the same shade of red and the background is kept at the same shade of grey. The saturation of the item 30 is changed (e.g. reduced) over time, for example as the item 30 moves, or in pauses between movements.

The user 40 then provides input that is indicative of the movement of the item 30. For example, at least one of the user input devices 45 can comprise an eye monitor 45b, 45e that monitors movement of the user's eye. The processing system 10 can then determine whether the movement of the user's 40 eye is correlated with the movement of the item 30. It is not necessary to determine the exact location of the user's gaze. Instead, the processing system 10 and eye monitor 45b can be configured to simply monitor movement of the eye or eyes, e.g. to determine whether the eye is moving or not and, if so, the direction in which it is moving. This allows a determination of whether the eye movement is correlated with the movement of the item 30. A point at which the movement of the eye becomes uncorrelated with the movement of the item 30 is indicative of a loss of perception of the item 30 by the user 40. The saturation of the item 30 when the user 40 loses perception of the item 30 is used to provide a measurement of the extent of perception of saturation of that colour by the user 40. By using correlation of eye movements rather than the exact location on the display that the gaze of the eye is on, then the processing required and the time to identify point at which the user fails to distinguish between the item 30 and the background 35 can be reduced. This in turn can result in a testing system 5 that is more efficient and more accurate.

Alternatives to using an eye monitor 45b can be used, e.g. by simply requiring the user to follow the item 30 using a suitable user input device 45 such as a touchscreen 45c, mouse, trackpad, or the like, and determining when the user fails to follow the item 30. However, it will be appreciated that using user eye movement is less intrusive to the user and can give faster and more accurate identification of the point at which the user fails to distinguish between the item 30 and the background 35.

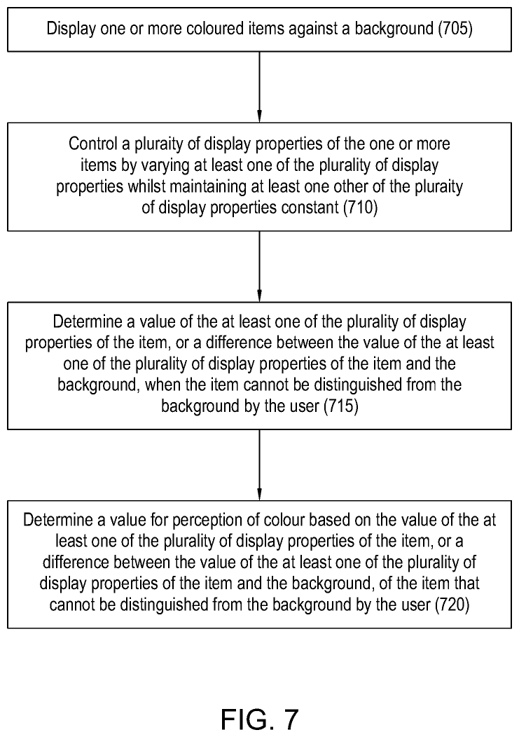

A summary of a method of determining and quantifying a user's perception of the at least one display property is shown in FIG. 7. This method can be performed by the systems 5, 5′ of FIG. 1 or 2 or another suitable testing system, and can apply to the techniques described in relation to any of FIGS. 3 to 6.

In step 705 one or more of the items 30 are displayed against a background 35, wherein at least one of the display properties (e.g. saturation) of the one or more items 30 differ from those of the background 35, and at least one of the display properties (e.g. luminance) of the one or more items 30 are the same as those of the background 35.

In step 710, the at least one of the plurality of display properties (e.g. saturation) of the at least one item 30 are varied whilst maintaining the at least one other of the plurality of display properties (e.g. hue and luminance) constant or successive items 30 are displayed for which the at least one of the plurality of display properties (e.g. saturation) differs but the at least one other of the plurality of display properties (e.g. hue and luminance) remains the same.

In step 715, a point at which the user fails to be able to distinguish the at least one item 30 being displayed from the background is identified and a value of the at least one of the plurality of display properties (e.g. saturation) of the at least one item 30, or a difference between the value of the at least one of the plurality of display properties of the item and the background, is determined for the item 30 that is being displayed at that point at which the user fails to be able to distinguish the at least one item 30 from the background. In step 720, the value of the at least one of the plurality of display properties (e.g. saturation) determined in step 715 is provided or used to determine a measure or value of the user's ability to perceive or sensitivity to the at least one display property (e.g. red saturation) for the user 40.

It will be appreciated that display of the items 30 and background 35 with the required display parameters on the visual display unit 25, 25′ is important. In order to do this, beneficially, the present inventors have developed a model based approach to generate colours with the same luminance, and for which an objective, simultaneous, and meaningful description of hue, saturation, and luminance can be produced. This includes, but is not limited to, red (for the items 30) and grey (for the background 35), or vice versa.

In the model, as known in the art, colour is described using a triplet of three values, R, G, and B, normalised to the range [0, 1], describing an additive colour model whereby colours are produced by superimposing red, green, and blue light, with intensities proportional to the R, G, and B values. When mapped to specific choices for the meaning of “red”, “green” and “blue”, this known colour model, called RGB, is well-suited to describe, amongst other things, the colour of the light emitted by a computer screen. When these specific choices are specified, for example utilising the description provided by the known CIELUV specification, this colour model becomes an absolute colour space. An example of absolute colour space, commonly used for computer screens, is SRGB [https://www.w3.org/Graphics/Color/sRGB.html].

From the RGB colour model, a “hue” H can be defined as:

$$H = \{ \begin{matrix}60 \times \frac{G - B}{Max ( R , G , B ) - Min ( R , G , B )} & if Max ( R , G , B ) = R \\ 60 \times ( 2 + \frac{B - R}{Max ( R , G , B ) - Min ( R , G , B )} ) & if Max ( R , G , B ) = G \\ 60 \times ( 4 + \frac{R - G}{Max ( R , G , B ) - Min ( R , G , B )} ) & if Max ( R , G , B ) = B\end{matrix}$$

A “luminance” Y is defined as

$$Y = {a}_{R} R + {a}_{G} G + {a}_{B} B$$

Wherein coefficients a_{R}, a_{G}, and a_{B}, depend on the absolute colour space chosen. These values are defined by international or other standard, and depend on the colour space used. For example, for the sRGB colour space, according to the WCAG 2.x standard, a_{R}=0.2126, a_{G}=0.7152, and a_{B}=0.0722. It must be noted that this system can be adapted to other colour spaces and standards by changing the coefficients a_{R}, a_{G}, and a_{B} accordingly.

In the model created by the present inventors, “saturation” S_{Y} can be defined from these definitions for hue H and luminance Y. In order to do so, each component of a given colour in the RGB model (normalised to the range [0,1]) is considered to be composed of a baseline grey level of RGB coordinates (h, h, h) where h is given by the relationship:

$$h = min ( R , G , B )$$

plus an excess red level r, an excess green level g, and an excess blue level b such that

$$r = R - h$$

$$g = G - h$$

$$b = B - h$$

At least one of the three excesses r, g, b is zero, due to the definition of h. The luminance of the baseline grey level Yₕ can be calculated according to the formula defining luminance Y given above, and is h. For example, when working in the sRGB colour space:

$${Y}_{h} = 0.2126 h + 0.7152 h + 0.0722 h = h$$

This luminance for the baseline grey level is independent from the colour space and luminance model chosen, as long as a_{R}+a_{G}+a_{B}=1.

Following this, the definition for saturation S_{Y} is

$${S}_{Y} = \{ \begin{matrix}1 - \frac{{Y}_{h}}{Y} = 1 - \frac{min ( R , G , B )}{Y} & if Y > 0 \\ 0 & if Y = 0\end{matrix}$$

In summary, the exemplary colour model, which can be referred to as HS_{Y}Y, is calculated from the R, G, and B components as:

$$H = \{ \begin{matrix}60 \times \frac{G - B}{Max ( R , G , B ) - Min ( R , G , B )} & if Max ( R , G , B ) = R \\ 60 \times ( 2 + \frac{B - R}{Max ( R , G , B ) - Min ( R , G , B )} ) & if Max ( R , G , B ) = G \\ 60 \times ( 4 + \frac{R - G}{Max ( R , G , B ) - Min ( R , G , B )} ) & if Max ( R , G , B ) = B\end{matrix}$$

$$Y = {a}_{R} R + {a}_{G} G + {a}_{B} B$$

$${S}_{Y} = \{ \begin{matrix}1 - \frac{min ( R , G , B )}{Y} & if Y > 0 \\ 0 & if Y = 0\end{matrix}$$

where the coefficients a_{R}, a_{G}, and a_{B}, depend on the absolute colour space and related luminance model chosen.

The inverse conversion (HS_{Y}Y to RGB) is mathematically more complex, but it can nevertheless be computed, for example using numerical algorithms, such as gradient descent and/or the like or by other suitable mathematical techniques.

When working with electronic displays, such as computer, mobile phone, or tablet monitors, the R, G, and B components cannot be sent to the electronic display directly, and an additional step is necessary to reproduce correctly a colour with known H, S_{Y}, and Y components. This is due to the fact that, for an electronic display, the red, green, and blue light intensity emitted by the display is not proportional to the values of R, G, and B sent to the display respectively but, rather, it is encoded through a non-linear display input/output characteristic. Hence, in order to obtain the correct values for H, S_{Y}, and Y from the display, it is necessary to send to the display values R′, G′, and B′ for the red, green, and blue components, that have been calculated from the desired values for R, G, and B by applying the inverse transformation to the display input/output characteristic. This results in the luminance of the red, green, and blue components of the light emitted by the display being linearly proportional to the R, G, and B values.

As an example, if the R, G, and B values correspond to the sRGB colour space, a computationally efficient approximation of the inverse of the display input/output characteristic often used for commercial monitors is, for the red component,

$${R}^{′} = \{ \begin{matrix}12.92 R & if R \leq 0.0031308 \\ 1.055 {R}^{\frac{1}{2.4}} - 0.055 & if R > 0.0031308\end{matrix}$$

For the G′ and B′ components, the same formula applies, with due replacement of R and R′ with G and G′, and B and B′, respectively.

The R′, G′, and B′ values are then sent to the display to obtain the colours with the desired R, G, and B values and, consequently, the respective H, S_{Y}, and Y values.

This formula is only a specific example that assumes a generic display input/output characteristic historically known as “gamma compression” or “gamma correction” or “gamma encoding” and which approximates a gamma curve with an overall gamma value of 2.2, which is not referred to any specific display model and/or setting, and that is approximated to be computationally efficient. While this example is therefore useable as a generic approximation for the inverse of the display input/output characteristic, any formula that constitutes an exact inversion or a good approximation of such inverse can be used as a replacement for the above formula for R′ (and the corresponding formulae for G′, and B′).

In view of the above, the present inventors have developed a set of tests, in which red targets on a grey background are displayed on a computer screen, and the target colour and the background are generated so that the hue H and luminance Y match, and only the saturation S_{Y} differs, as illustrated above in relation to FIGS. 3 to 6. A test subject attempts to detect or differentiate items 30 from the background 35 or between each other, and the value(s) of S_{Y} the test subject detects/identifies the item 30 can be determined.

With this, it is possible to provide a quantitative and objective saturation test.

Although various examples are described above, variations to the above procedures and apparatus are possible.

For example, references made herein are to red saturation. However, by changing the hue, this test is not restricted to the colour red.

In addition, the formulas given allow defining, for every colour, a hue, a saturation, and a luminance. As such, the approaches described herein can be used to measure a wide range of perception parameters.

The techniques described herein can be used to determine saturation perception of a range of colours, of which red desaturation diagnostics is merely an example, by modifying the saturation of the items (i.e. the shapes, or optotypes etc.) with respect to the background or to each other, maintaining fixed the hue and the luminance.

Techniques analogous to those described herein can also be used to perform luminance perception diagnostics, by modifying the luminance of the items 30 (e.g. shapes or optotypes) with respect to the background or to each other, by maintaining fixed the hue and the saturation and varying the luminance. In this case, the saturation is beneficially non-zero and the method is more effective when the items 30 are moving.

Techniques analogous to those described herein can also be used to perform hue perception diagnostics, by modifying the hue of the shapes with respect to the background or to each other, whilst maintaining fixed luminance and saturation. The model described above allows for the development of tests in which the matching of luminance and saturation is guaranteed. The use of different items 30 in the form of shapes and optotypes provide a more general family of tests that can be rigorously designed in all their parameters. Having the items 30 moving also allows for more accurate and convenient automated identification of the point at which the user ceases to be able to identify the items 30 from the background 35.

By setting hue, saturation and luminance, of static patterns, by changing the size of the patterns, and exploring the visibility of patterns of different sizes, the approaches described herein allow the measurement and diagnosis of static visual acuity for different values of hue, saturation, and luminance, with each of these parameters rigorously defined.

By setting hue, saturation and luminance, of moving patterns, by changing the size of the patterns, and exploring the visibility of patterns of different sizes, the approaches described herein allow the measurement and diagnosis of dynamic visual acuity for different values of hue, saturation, and luminance, with each of these parameters rigorously defined.

By setting hue, saturation and luminance, of moving patterns, by changing the speed of the patterns, and exploring the visibility of patterns of different speeds, the approaches described herein allow the measurement and diagnosis of the visibility of the patterns for different values of hue, saturation, luminance, and speed, with each of these parameters rigorously defined.

The systems identified in FIGS. 1 and 2 are merely provided as examples of the systems that can implement the approaches described herein, and it will be appreciated that the techniques described herein can be implemented with other systems having processing and display capability.

Method steps of the invention can be performed by one or more programmable processors executing a computer program to perform functions of the invention by operating on input data and generating output. Method steps can also be performed by special purpose logic circuitry, e.g., an FPGA (field programmable gate array) or an ASIC (application-specific integrated circuit) or other customised circuitry. Processors suitable for the execution of a computer program include CPUs and microprocessors, and any one or more processors. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, e.g. EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in special purpose logic circuitry.

To provide for interaction with a user, the invention can be implemented on a device having a screen, e.g., a CRT (cathode ray tube), plasma, LED (light emitting diode) or LCD (liquid crystal display), or OLED (organic LED) monitor, or a projector, e.g. a projection system based on LCD, or on a DLP (digital light processing) array, or on a LCOS (liquid crystal on silicon) chip, for displaying information to the user and an input device, e.g., a keyboard, touch screen, a mouse, a trackball, and the like by which the user can provide input to the computer. Other kinds of devices can be used, for example, feedback provided to the user can be any form of sensory feedback, e.g., visual feedback, auditory feedback, or tactile feedback; and input from the user can be received in any form, including acoustic, speech, or tactile input. Input can be either voluntary (i.e., the user deliberately provides the input), or involuntary (i.e. a measuring device such as e.g. an accelerometer, or an eye tracker, or a camera measures a user response, e.g. a reflex, that does not require the user needing to make a deliberate action to provide the input). Operating on involuntary inputs may be beneficial when the user cannot provide a voluntary input, e.g. because they are too young to understand or obey to instructions, because they are cognitively impaired, etc.

As such, while certain embodiments have been described, these embodiments have been presented by way of example only, and are not intended to limit the scope of the invention. Indeed the novel methods and systems described herein may be embodied in a variety of other forms. Furthermore, various omissions, substitutions and changes in the form of the methods and systems described herein may be made without departing from the spirit of the invention. The accompanying claims and their equivalents are intended to cover such forms and modifications as would fall within the scope of the invention.

1. A computer implemented testing system configured to control at least one visual display system to:
display one or more items against a background, wherein at least one display property of the one or more items differs from that of the background and at least one other display property of the one or more items is the same as that of the background; and
vary the at least one display property of the one or more items; or
display different items or groups of items of the one or more items wherein the at least one display property differs between different items or groups of items.

2. The testing system of claim 1, wherein the display properties comprise at least two or all of: luminance, hue and/or saturation.

3. The testing system of claim 2, wherein:
the at least one display property comprises saturation; and
the at least one other display property comprises luminance and/or hue.

4. The testing system of claim 3, configured to successively reduce or increase the saturation of successively displayed items or to reduce or increase the saturation of the one or more items with time.

5. The testing system of claim 4, configured to switch or alternate between increasing and decreasing the saturation of successively displayed items or to switch or alternate between increasing and decreasing the saturation of the one or more items with time.

6. The testing system of claim 1, configured to:
provide successively displayed items of the one or more items, with each successively displayed item having a value for the one or more display properties that is closer to or further from that of the background than a previously displayed item; or
vary the one or more display properties of the one or more items over time to be closer to that of the background.

7. The testing system of claim 1, configured to:
provide successively displayed items of the one or more items, wherein the testing system is configured to switch or alternate between:
providing one or more of the successively displayed items having a value for the one or more display properties that is further from that of the background than a previously displayed item; and
providing one or more of the successively displayed item having the value for the one or more display properties that is closer to that of the background than a previously displayed item;
or
switch or alternate between varying the one or more display properties of the one or more items over time to be further from that of the background and varying the one or more display properties of the one or more items over time to be closer to that of the background.

8. The testing system of claim 7 configured to:
provide the successively displayed items or vary the one or more display properties at least until the user is able or unable to distinguish the displayed item or items from the background; and
determining a value for the users' ability to perceive the at least one display property comprising or based on the determined value of the at least one display property with which the at least one item is being displayed when the user is able or unable to distinguish the at least one item displayed from the background.

9. The testing system of claim 1, wherein the background is of a different colour to the one or more items.

10. The testing system according to claim 7, wherein the items are coloured red and the background is coloured grey.

11. The testing system of claim 10, wherein:
the at least one display property of the of the one or more items that is varied or differs between items or groups of items includes saturation; and the other display properties of the one or more items that are the same as those of the background include luminance and hue.

12. The testing system of claim 1, wherein the visual display system comprises a headset display.

13. The testing system of claim 1, comprising or configured to communicate with a user input device, the user input device being configured to receive user input indicative of the user's perception of the displayed items, wherein the testing system is configured to:
identify when the user input indicative of the user's perception of the displayed items differs from, or starts to become correlated with, the items being displayed;
determine the value of the at least one display property with which the at least one item is being displayed when it is identified that the user input indicative of the user's perception of the displayed items differs from, or starts to become correlated with, the items being displayed; and
provide a value for the users' ability to perceive the at least one display property comprising or based on the determined value of the at least one display property with which the at least one item is being displayed when it is identified that the user input indicative of the user's perception of the displayed items differs from or becomes the same as the items being displayed.

14. The testing system of claim 13, wherein the user input device is a passive user input device for sensing passive user input.

15. The testing system of claim 11, wherein the display is configured to move the at least one item, and wherein the user input device is configured to receive user input indicative of the movement or location of the at least one item and optionally, the user input device comprises one or more from: a motion tracker of a headset, at least one accelerometer, a gyroscope, a magnetometer, and/or at least one camera.

16. The testing system of claim 13, wherein the user input device comprises an eye and/or head movement tracker for tracking movement of a users' eye and/or head, the testing system being configured to:
determine when the movement of the user's eye and/or head ceases or begins to be correlated with the position and/or movement of the movement of the at least one item;
determine the value of the at least one display property with which the at least one item is being displayed when the position and/or movement of the user's eye and/or head ceases or begins to be correlated with the position and/or movement of the movement of the at least one item; and
provide a value for the users' ability to perceive the at least one display property comprising or based on the determined value of the at least one display property with which the at least one item is being displayed when the position and/or movement of the user's eye and/or head ceases or begins to be correlated with the position and/or movement of the movement of the at least one item.

17. The testing system of claim 16, wherein one or more or each of:
the determination of when the movement of the user's eye and/or head ceases or begins to be correlated with the position and/or movement of the movement of the at least one item;
the determination of the at least one display property with which the at least one item is being displayed when the position and/or movement of the user's eye and/or head ceases or begins to be correlated with the position and/or movement of the movement of the at least one item; and/or
the determination and/or provision of the value for the users' ability to perceive the at least one display property is performed offline and/or not during the test and/or by a remote computer that is remote from the user input device that is optionally connected to the user input device via a wide area network.

18. The system of claim 11, wherein a plurality of the items or groups of items are provided, each having a different property, and the user input device is configured to receive an indication of the user's perception of the property of the item being displayed.

19. The system of claim 18, wherein the properties comprise at least one of: a shape of the item, an optotype, number or letter of an item, a size of the item, a number of items in the group of items, a location of the item, the presence of movement, the speed of movement, a change in shape, a change in size, and/or a direction of movement of the item.

20. The system of claim 13, comprising identifying a condition of the user based on the value for the users' ability to perceive the at least one display property.

21. The system of claim 20, wherein the condition is a condition of the eye, optic nerve, or visual processing part of brain.

22. The testing system of claim 1, comprising a colour model defining at least one or each of the display properties of the item based on red, green and blue values for the colour.

23. The testing system of claim 22, wherein the colour model defines at least one or each of the display properties of the item based on a baseline grey level or a grey level of the background modified by altering the at least one other of the display properties.

24. The testing system of claim 23, wherein the colour model defines the saturation of the item based on a baseline grey level or a grey level of the background modified by altering one or two amongst the red, green, and blue values for the colour.

25. A method of operating the testing system of claim 1, the method comprising operating the testing system to:
display one or more items against a background, wherein at least one display property of the one or more items differs from that of the background and at least one other display property of the one or more items is the same as that of the background; and
vary the at least one display property of the one or more items; or
display different items or groups of items of the one or more items wherein the at least one display property differs between different items or groups of items.

26. A computer program product configured such that, when executed by the testing system of claim 1, causes the testing system to;
display one or more items against a background, wherein at least one display property of the one or more items differs from that of the background and at least one other display property of the one or more items is the same as that of the background; and
vary the at least one display property of the one or more items; or
display different items or groups of items of the one or more items wherein the at least one display property differs between different items or groups of items.
